SEQUENCE LISTING (9 sequences)

SEQ ID NO: 1 - DNA, 94 bp, Artificial Sequence
  Synthetic
  misc_feature (49)..(49): n is inosine
  misc_feature (52)..(52): n is inosine
  misc_feature (55)..(55): n is inosine
  misc_feature (58)..(58): n is inosine
ctgtctcttatacacatctgacgctgccgacgattttcctcctcctccnttnttnttnttgtctcgtgggctcggagatgtgtataagagacag

SEQ ID NO: 2 - DNA, 14 bp, Artificial Sequence
  Synthetic
gacgctgccgacga

SEQ ID NO: 3 - DNA, 15 bp, Artificial Sequence
  Synthetic
gtctcgtgggctcgg

SEQ ID NO: 4 - DNA, 27 bp, Artificial Sequence
  Synthetic
  misc_feature (16)..(16): n is inosine
  misc_feature (19)..(19): n is inosine
  misc_feature (22)..(22): n is inosine
  misc_feature (25)..(25): n is inosine
ttttcctcctcctccnttnttnttntt

SEQ ID NO: 5 - DNA, 19 bp, Artificial Sequence
  Synthetic
agatgtgtataagagacag

SEQ ID NO: 6 - DNA, 19 bp, Artificial Sequence
  Synthetic
ctgtctcttatacacatct

SEQ ID NO: 7 - DNA, 55 bp, Artificial Sequence
  Synthetic
gcctggagctccgttttttttaacggagctccaggcagatgtgtataagagacag

SEQ ID NO: 8 - DNA, 94 bp, Artificial Sequence
  Synthetic
  misc_feature (49)..(60): n can be thymine, inosine, uridine, 5-methylcytosine, isoguanine, 2-thiouracil, or 4-thiouracil
ctgtctcttatacacatctgacgctgccgacgattttcctcctcctccnnnnnnnnnnnngtctcgtgggctcggagatgtgtataagagacag

SEQ ID NO: 9 - DNA, 74 bp, Artificial Sequence
  Synthetic
ctgtctcttatacacatctgcctggagctccgttttttttaacggagctccaggcagatgtgtataagagacag

(19) United States
(12) Patent Application Publication
(10) Pub. No.: US 2022/0356518 A1
(43) Pub. Date: Nov. 10, 2022

(54) UNIVERSAL ADAPTOR FOR SEQUENCING

(71) Applicant: Quantum-Si Incorporated, Guilford, CT (US)

(72) Inventors: Jeffrey Sabina, Hamden, CT (US); Jonathan M. Rothberg, Miami Beach, FL (US); John H. Leamon, Stonington, CT (US)

(73) Assignee: Quantum-Si Incorporated, Guilford, CT (US)

(21) Appl. No.: 17/733,888

(22) Filed: Apr. 29, 2022

Related U.S. Application Data
(60) Provisional application No. 63/182,604, filed on Apr. 30, 2021.

(51) Int. Cl. C12Q 1/6874; C12N 15/10; C12Q 1/6876

(52) U.S. Cl. CPC C12Q 1/6874; C12N 15/1065; C12Q 1/6876

(57) ABSTRACT

Methods and compositions for preparing nucleic acid libraries for nucleic acid sequencing are provided. In some embodiments, disclosed herein is a universal nucleic acid adaptor and methods of using same.

### CROSS-REFERENCE TO RELATED APPLICATION

This application claims priority under 35 U.S.C. § 119(e) to U.S. Provisional Patent Application No. 63/182,604, filed Apr. 30, 2021, which is hereby incorporated by reference in its entirety.

### BACKGROUND OF INVENTION

Nucleic acid sequencing (e.g., next-generation sequencing (NGS)) requires upfront preparation of library molecules from biological sources. The particular steps, requirements and methods are specific to each sequencing platform, but generally, library preparation processes fragment or amplify regions of nucleic acids from a biological sample, and then add nucleic acid sequences on the ends of the fragments via PCR, ligation, or other means, in order to facilitate downstream amplification and/or sequencing. However, these library preparation processes can be challenging to implement. Accordingly, novel compositions and methods for library preparation are needed.

### SUMMARY OF INVENTION

Aspects of the instant disclosure provide methods and compositions for nucleic acid sequencing. In some aspect, the disclosure provides a universal nucleic acid adaptor (e.g., for use across, or compatible with multiple sequencing platforms). In some embodiments, a universal nucleic acid adaptor comprises a double-stranded transposase recognition sequence; a first primer-binding sequence; and a pair of index primer-binding sequences comprising a first index primer-binding sequence and a second index primer-binding sequence.

In some embodiments, the double-stranded transposase recognition sequence is a double-stranded Tn5 transposase recognition sequence. In some embodiments, the double-stranded transposase recognition sequence is a mosaic end. In some embodiments, the double-stranded transposase recognition sequence is 15-25 nucleotides in length (e.g., 17-23, 18-22, 19-21, or 19 19 nucleotides in length). In some embodiments, a first strand of the double-stranded transposase recognition sequence comprises the nucleic acid sequence set forth in SEQ ID NO: 5 and/or SEQ ID NO: 6. In some embodiments, the first strand of the double-stranded transposase recognition sequence comprises the nucleic acid sequence set forth in SEQ ID NO: 5, and a second strand of the double-stranded transposase recognition sequence comprises the nucleic acid sequence set forth in SEQ ID NO: 6.

In some embodiments, the first primer-binding sequence comprises one or more non-standard nucleotides selected from the group consisting of: inosine, uridine, 5-methylcytosine, isoguanine, 2-thiouracil, and 4-thiouracil. In some embodiments, the first primer-binding sequence comprises four inosine nucleotides.

In some embodiments, the first primer-binding sequence comprises the nucleic acid sequence set forth in SEQ ID NO: 4. In some embodiments, the first index primer-binding sequence comprises the nucleic acid sequence set forth in SEQ ID NO: 2 and/or SEQ ID NO: 3.

In some embodiments, the first primer-binding sequence is configured for use in a first sequencing instrument, and the pair of index primer-binding sequences are configured for use in a second sequencing instrument. In some embodiments, the first sequencing instrument is a long-read sequencing instrument. In some embodiments, the first sequencing instrument is a high-throughput sequencing instrument.

In some embodiments, the universal nucleic acid adaptor comprises the nucleic acid sequence set forth in SEQ ID NO: 1. In some embodiments, the universal nucleic acid adaptor comprises the nucleic acid sequence set forth in SEQ ID NO: 8.

Some aspects of the disclosure provide a circular nucleic acid comprising one or two nucleic acid adaptors, wherein each nucleic acid adaptor is independently selected from any one of the adaptors (e.g., universal adaptors) described herein. In some embodiments, the circular nucleic acid comprises two nucleic acid adaptors on opposite sides of the circular nucleic acid. In some embodiments, the circular nucleic acid comprises two identical nucleic acid adaptors.

Some aspects of the disclosure provide a method of preparing a nucleic acid library for sequencing. In some embodiments, a method of preparing a nucleic acid library for sequencing comprises: (i) contacting a target nucleic acid with a transposon and any one of the adaptors (e.g., universal adaptors) described herein to generate a transposase-mediated fragment; and (ii) contacting the transposase-mediated fragment with one or more enzymes necessary to fill the gaps and circularize the fragment. In some embodiments, a method of preparing a nucleic acid library for sequencing comprises: (i) contacting a target nucleic acid with a transposon and any one of the circular nucleic acids described herein to generate a transposase-mediated fragment; and (ii) contacting the transposase-mediated fragment with one or more enzymes necessary to fill the gaps and circularize the fragment.

In some embodiments, the transposon is a member of the Tn5 transposase family of proteins, optionally wherein the transposon is a Tn5 transposase. In some embodiments, the one or more enzymes necessary to fill the gaps and circularize the fragment comprise a polymerase and a ligase. In some embodiments, the target nucleic acid is from a biological sample, optionally wherein the biological sample is a blood, saliva, sputum, feces, urine, nasal, mucus, or buccal swab sample.

### BRIEF DESCRIPTION OF DRAWINGS

FIG. 1 provides an example of a universal nucleic acid adaptor of the disclosure (SEQ ID NO: 1) and an example process for the production of a circular nucleic acid comprising said nucleic acid adaptor.

FIGS. 2A-2C provide example methods for the production of nucleic acid libraries using an example circular nucleic acid of the disclosure. FIG. 2A shows a method comprising primer annealing to, and extension from the first primer-binding sequence of the nucleic acid adaptor. FIG. 2B shows a method comprising endonuclease V digestion of inosine nucleotides in the first primer-binding sequence of the nucleic acid adaptor, followed by generation of blunt ends using an exonuclease, and ligation of motor proteins (e.g., helicases, polymerases) to the blunt ends of the adaptors. FIG. 2C shows a method comprising primer annealing to, and extension from both index primer-binding sequences of the nucleic acid adaptor, followed by subsequent primer annealing to, and extension from either index primer-binding sequence.

FIG. 3 provides an example method of transposon generation of linear fragment libraries. A nucleic acid sequence (“QsiTN5a ver1”; SEQ ID NO: 7), a Tn5 mosaic end (SEQ ID NO: 5), and a Tn5 mosaic end—reverse (SEQ ID NO: 6) are loaded onto a transposome. The loaded transposome is subsequently utilized to mediate fragmentation of a target nucleic acid and ultimately generate linear fragment libraries.

FIG. 4 provides example methods of circularization of nucleic acids. Methods include circularization using a blunt ended hairpin payload (left panel), circularization using an overhang hairpin and non-phosphorylated squib payload (middle panel) and circularization using an overhang hairpin and phosphorylated squib payload (right panel).

FIG. 5 provides a schematic diagram of a cross-section view of a cartridge 100 along the width of channels 102, in accordance with some embodiments.

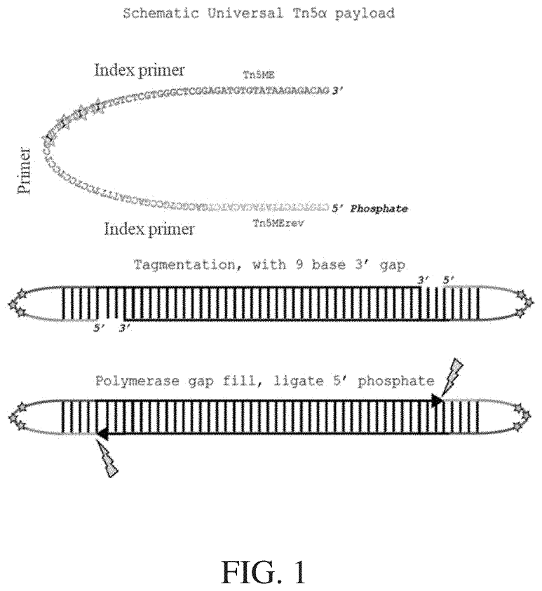

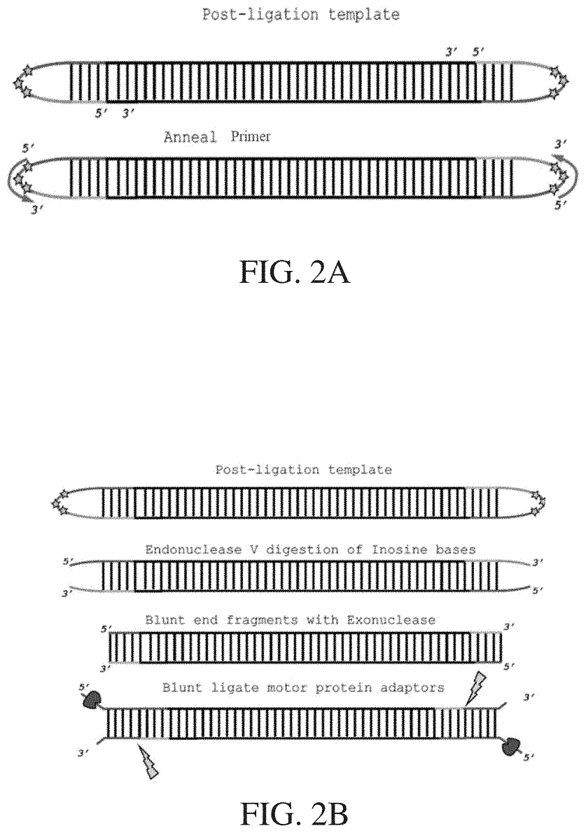

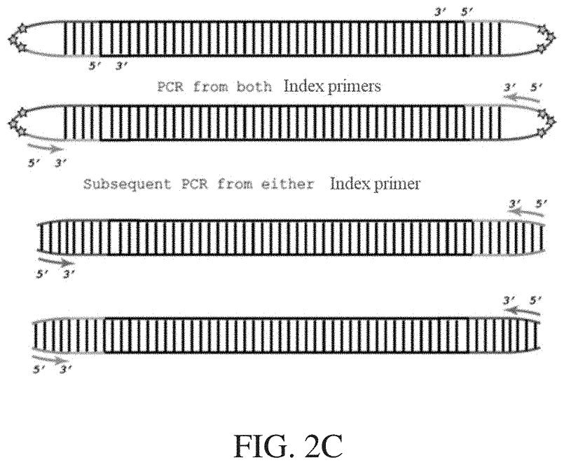

### DETAILED DESCRIPTION OF INVENTION

Herein, the disclosure provides compositions and methods to enable the use of a single next generation sequencing (NGS) adaptor molecule. Furthermore, the disclosure provides a universal nucleic acid adaptor capable of being used across several (or all) nucleic acid sequencing platforms. The universal nucleic acid adaptors described herein satisfy the structural and sequence requirements across each of these nucleic acid sequencing platforms.

Additionally, the universal nucleic acid adaptors described herein provide substantial improvements on existing “short read” PCR-driven sample preparation processes because 100% of the generated product is amplifiable (e.g., flanked by heterogenous A & B primers), as opposed to traditional practices (that do not utilize universal nucleic acid adaptors as described) that produce generated products only 50% of which are amplifiable. The universal adaptor also provides a uniform methodology for which both long, multi-kilobase and shorter, sub-kilobase reads can be generated from a single DNA sample, providing both a valuable scaffold for de novo assembly as well as deep short read coverage to ensure high consensus sequence accuracy.

The initial step in many library preparation workflows is to break up genomic DNA into fragment sizes appropriate to the target nucleic acid sequencing platform. This can be accomplished by several means. In some embodiments, this fragmentation is done using tagmentation, a near random enzymatic fragmentation of the DNA by a transposon (e.g., a member of the Tn5 transposase family of proteins such as Tn5 transposase) which results in DNA of lower length with the addition of short DNA sequences on either side of the cut site. To accomplish this, the Tn5 protein requires assembly with a minimal double-stranded DNA sequence called a Mosaic End (or ME) sequence. Addition of DNA to the non-invading end of the duplex is quite permissible, allowing the addition of long stretches of dsDNA as well as looped and unpaired strands, which enables the generation of the universal adaptors described herein which comprise a first primer sequence and a pair of index primer binding sites comprising a first index primer sequence and a second index primer sequence (e.g., a first primer sequence that is configured for use in a first sequencing instrument and a pair of index primer binding sites that are configured for use in a second sequencing instrument).

The requirements for nucleic acid adaptors generally fall into one of two categories: 1) structural features; and 2) sequence features. For example, long-read platforms perform sequencing directly from adapted DNA fragments and require no amplification. In some embodiments, the adaptor must form a single-stranded loop structure at the end of the linear double-stranded DNA and must contain a binding site with sequence complementary to a sequencing primer. In some embodiments, an adaptor is added that is pre-loaded with a motor protein via ligation to blunt-ended, fragmented DNA. In short-read platforms, there are no specific structural requirements (typical adaptors used are simple double-stranded DNA molecules), but there is a requirement for binding sites complementary to primers that can be used to amplify the adapted fragments or to amplify using tailed primers to both amplify and add additional sequences to each fragment to accommodate amplification in templating and/or additional binding sites for sequencing primers of other features.

In some embodiments, the nucleic acid adaptor comprises a single Tn5-compatible adaptor, containing a stem-loop structure, which contains binding sites complementary to a first sequencing primer, as well as sites enabling amplification and indexing using the indexing primers. By including a pair of non-complementary, indexing primer binding sites (e.g., Illumina i7 and i5 sequences) in the non-base paired portion of the adaptor, it allows for each strand of the DNA fragment to be linked to a different primer binding site. In this way, a single DNA fragment resulting from Tn5 symmetric insertion and tagging on both sides can be labeled in a way that enables asymmetric addition of primer sites to each strand. This design takes advantage of the unpaired loop structure to house elements required for long and short-read platforms in a single Tn5-adaptor assembly, which creates a seal-ended dsDNA fragment after tagmentation and gap closure. Using a single adaptor provides benefits in manufacturing as only one molecule needs to be manufactured, and through the use of unpaired regions to add binding sites asymmetrically, avoids creation of non-productive fragments that would result when two different species of adaptor are used and all combinations of A and B end addition are present (A-A; B-B; A-B; B-A) in addition to the desired asymmetric molecule (i.e., A-B and B-A).

An added feature of incorporating a nucleotide not typically present in natural genomic DNA that can be specifically be cleaved (via enzymatic or chemically processes) (e.g., deoxy-Uridine, deoxy-Inosine, RNA bases, etc.) in the loop region would allow for separation of the top and bottom strands during subsequent PCR amplification steps if required.

In some embodiments, the nucleic acid adaptor described herein provides several benefits over traditional adaptor approaches. In some embodiments, these benefits include that the nucleic acid adaptor described herein is universal, automatable, and enables a streamlined workflow to supply libraries for multiple sequencing platforms. In some embodiments, these benefits include that the nucleic acid adaptor described herein is universal, automatable, and enables a workflow to supply both multi-kilobase and sub-kilobase reads from a single DNA source, allowing long read scaffolding and short-read variant detection from the same material. In some embodiments, these benefits include that the nucleic acid adaptor described herein generates 100% PCR amplifiable material, rather than 50% as produced by traditional methodologies.

The term “nucleic acid,” as used herein, generally refers to a molecule comprising one or more nucleic acid subunits. A nucleic acid may include one or more subunits selected from adenine (A), cytosine (C), guanine (G), thymine (T), uracil (U), inosine (I), and any modified nucleobase or variant thereof. In some examples, a nucleic acid is deoxyribonucleic acid (DNA) or ribonucleic acid (RNA), or derivatives thereof. A nucleic acid may be single-stranded or double-stranded. A nucleic acid may be circular.

The term “nucleic acid adaptor” denotes a nucleic acid that can be used for manipulation or altering or handling of a target nucleic acid. In some embodiments, a nucleic acid adaptor can be used for circularization of a target nucleic acid. In some embodiments, a nucleic acid adaptor can be used to introduce a nick or gap in a target nucleic acid. In some embodiments, a nucleic acid adaptor can be used to facilitate sequencing methods for sequencing a target nucleic acid or a segment thereof. In some embodiments, a nucleic acid adaptor can have one or more ends that lack a 5′ phosphate residue. In some embodiments, a nucleic acid adaptor comprises a single-stranded nucleic acid. In some embodiments, a nucleic acid adaptor as described herein comprises two or more one primer-binding sequences.

The term “nucleotide,” as used herein, generally refers to a nucleic acid subunit, which can include A, C, G, T, U, I, or variants or analogs thereof. A nucleotide can include any subunit that can be incorporated into a growing nucleic acid strand. Such subunit can be an A, C, G, T, U, I, or any other subunit that is specific to one or more complementary A, C, G, T, U, I, or complementary to a purine (e.g., A or G, or variant or analogs thereof) or a pyrimidine (e.g., C, T, U, I, or variant or analogs thereof). A subunit can enable individual nucleic acid bases or groups of bases (e.g., AA, TA, AT, GC, CG, CT, TC, GT, TG, AC, CA, or uracil-counterparts thereof) to be resolved.

A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate, which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, that include detectable tags, such as luminescent tags or markers (e.g., fluorophores).

A nucleoside polyphosphate can have ‘n’ phosphate groups, where ‘n’ is a number that is greater than or equal to 2, 3, 4, 5, 6, 7, 8, 9, or 10. Examples of nucleoside polyphosphates include nucleoside diphosphate and nucleoside triphosphate. A nucleotide can be a terminal phosphate labeled nucleoside, such as a terminal phosphate labeled nucleoside polyphosphate. Such label can be a luminescent (e.g., fluorescent or chemiluminescent) label, a fluorogenic label, a colored label, a chromogenic label, a mass tag, an electrostatic label, or an electrochemical label. A label (or marker) can be coupled to a terminal phosphate through a linker. The linker can include, for example, at least one or a plurality of hydroxyl groups, sulfhydryl groups, amino groups or haloalkyl groups, which may be suitable for forming, for example, a phosphate ester, a thioester, a phosphoramidate or an alkyl phosphonate linkage at the terminal phosphate of a natural or modified nucleotide. A linker can be cleavable so as to separate a label from the terminal phosphate, such as with the aid of a polymerization enzyme. Examples of nucleotides and linkers are provided in U.S. Pat. No. 7,041,812, which is entirely incorporated herein by reference.

A nucleotide (e.g., a nucleotide polyphosphate) can comprise a methylated nucleobase. For example, a methylated nucleotide can be a nucleotide that comprises one or more methyl groups attached to the nucleobase (e.g., attached directly to a ring of the nucleobase, attached to a substituent of a ring of the nucleobase). Exemplary methylated nucleobases include 1-methylthymine, 1-methyluracil, 3-methyluracil, 3-methylcytosine, 5-methylcytosine, 1-methyladenine, 2-methyladenine, 7-methyladenine, N6-methyladenine, N6,N6-dimethyladenine, 1-methylguanine, 7-methylguanine, N2-methylguanine, and N2,N2-dimethylguanine.

The term “primer-binding sequence,” as used herein, generally refers to nucleic acid sequence to which a primer can anneal or otherwise bind. In some embodiments, a primer-binding sequence is a nucleic acid sequence to which a primer anneals and causes polymerization and/or extension. A primer can be a synthetic oligonucleotide comprising DNA, RNA, PNA, or variants or analogs thereof. A primer-binding sequence can be designed such that its nucleotide sequence is complementary to a primer, or the primer-binding sequence can comprise one or more mismatched pairs with a primer. In some embodiments, a primer-binding sequence can comprise 5 to 15 bases, 10 to 20 bases, 15 to 25 bases, 20 to 30 bases, 25 to 35 bases, 30 to 40 bases, 35 to 45 bases, 40 to 50 bases, 45 to 55 bases, 50 to 60 bases, 55 to 65 bases, 60 to 70 bases, 65 to 75 bases, 70 to 80 bases, 75 to 85 bases, 80 to 90 bases, 85 to 95 bases, 90 to 100 bases, 95 to 105 bases, 100 to 150 bases, 125 to 175 bases, 150 to 200 bases, or more than 200 bases.

### Sample Preparation

In some aspects, the disclosure provides processes for preparing a sample, e.g., for detection and/or analysis. In some embodiments, a process described herein may be used to identify properties or characteristics of a sample, including the identity or sequence (e.g., nucleotide sequence) of one or more target molecules in the sample. In some embodiments, a process may include one or more sample transformation steps, such as sample lysis, sample purification, sample fragmentation, purification of a fragmented sample, library preparation (e.g., nucleic acid library preparation), purification of a library preparation, sample enrichment (e.g., using affinity SCODA), and/or detection/analysis of a target molecule.

In some embodiments, a sample may be a purified sample, a cell lysate, a single-cell, a population of cells, or a tissue. In some embodiments, a sample is any biological sample. In some embodiments, a sample (e.g., a biological sample) is a blood, saliva, sputum, feces, urine, nasal, mucus or buccal swab sample. In some embodiments, a biological sample is from a human, a non-human primate, a rodent, a dog, a cat, a horse, or any other mammal. In some embodiments, a biological sample is from a bacterial cell culture (e.g., an E. coli bacterial cell culture). A bacterial cell culture may comprise gram positive bacterial cells and/or gram negative bacterial cells. In some embodiments, a sample is a purified sample of nucleic acids that have been previously extracted via user-developed methods from metagenomic samples or environmental samples. A blood sample may be a freshly drawn blood sample from a subject (e.g., a human subject) or a dried blood sample (e.g., preserved on solid media (e.g., Guthrie cards)). A blood sample may comprise whole blood, serum, plasma, red blood cells, and/or white blood cells.

In some embodiments, a sample (e.g., a sample comprising cells or tissue) may be prepared, e.g., lysed (e.g., disrupted, degraded and/or otherwise digested) in a process in accordance with the instant disclosure. In some embodiments, a sample to be prepared, e.g., lysed, comprises cultured cells, tissue samples from biopsies (e.g., tumor biopsies from a cancer patient, e.g., a human cancer patient), or any other clinical sample. In some embodiments, a sample comprising cells or tissue is lysed using any one of known physical or chemical methodologies to release a target molecule (e.g., a target nucleic acid) from said cells or tissues. In some embodiments, a sample may be lysed using an electrolytic method, an enzymatic method, a detergent-based method, and/or mechanical homogenization. In some embodiments, a sample (e.g., complex tissues, gram positive or gram negative bacteria) may require multiple lysis methods performed in series. In some embodiments, if a sample does not comprise cells or tissue (e.g., a sample comprising purified nucleic acids), a lysis step may be omitted. In some embodiments, lysis of a sample is performed to isolate target nucleic acid(s). In some embodiments, a lysis method further includes use of a mill to grind a sample, sonication, surface acoustic waves (SAW), freeze-thaw cycles, heating, addition of detergents, addition of protein degradants (e.g., enzymes such as hydrolases or proteases), and/or addition of cell wall digesting enzymes (e.g., lysozyme or zymolase). Exemplary detergents (e.g., non-ionic detergents) for lysis include polyoxyethylene fatty alcohol ethers, polyoxyethylene alkylphenyl ethers, polyoxyethylene-polyoxypropylene block copolymers, polysorbates and alkylphenol ethoxylates, preferably nonylphenol ethoxylates, alkylglucosides and/or polyoxyethylene alkyl phenyl ethers. In some embodiments, lysis methods involve heating a sample for at least 1-30 min, 1-25 min, 5-25 min, 5-20 min, 10-30 min, 5-10 min, 10-20 min, or at least 5 min at a desired temperature (e.g., at least 60° C., at least 70° C., at least 80° C., at least 90° C., or at least 95° C.).

In some embodiments, a sample is prepared, e.g., lysed, in the presence of a buffer system. This buffer system may be used to make a slurry of the sample, to suspend the sample, and/or to stabilize the sample during any known lysis methodology, including those methods described herein. In some embodiments, a sample is prepared, e.g., lysed, in the presence of RIPA buffer, GCI buffer that comprises Guanidine-HCl buffer, Gly-NP40 buffer, a TRIS buffer, a HEPES buffer, or any other known buffering solution.

Many of the lysis methods described herein allow for the sample to be lysed by mechanically homogenizing the sample such that the cell walls of the sample break down. For example, methods that cause lysis by mechanical homogenization include, but are not limited to, bead-beating, heating (e.g., to high temperatures sufficient to disrupt cell walls, e.g., greater than 50° C., 60° C., 70° C., 80° C., 90° C., or 95° C.), syringe/needle/microchannel passage (to cause shearing), sonication, or maceration with a grinder. In some embodiments, any lysis methodology may be combined with any other lysis methodology. For example, any lysis methodology may be combined with heating and/or sonication and/or syringe/needle/microchannel passage to quicken the rate of lysis.

In some embodiments, sample preparation comprises cell disruption (i.e., subsequent removal of unwanted cell and tissue elements following lysis). In some embodiments, cell disruption involves protein and/or nucleic acid precipitation. In some embodiments, following precipitation, the lysed and disrupted sample is subjected to centrifugation. In some embodiments, following centrifugation, the supernatant is discarded. Precipitation can be accomplished through multiple processes, including but not limited to those methods described in Winter, D. and H. Steen (2011). “Optimization of cell lysis and protein digestion protocols for the analysis of HeLa S3 cells by LC-MS/MS.” PROTEOMICS 11(24): 4726-4730. In some embodiments, proteins or peptides are immunoprecipitated. In some embodiments, centrifugation of precipitated proteins and/or nucleic acids is followed by discarding of the supernatant and subsequent washing of the pellet fraction (e.g., washing using chloroform/methanol or trichloroacetic acid).

In some embodiments, a sample is prepared using lysis in the presence of a lysis buffer (e.g., GCI buffer (6M Guanidine HCl, 0.1 M TEAB, 1% Triton X-100, a standard buffer, and 1 mM EDTA/EGTA)) and disrupted by needle shearing (e.g., by passage of the sample through a 26.5 gauge needle, e.g., at 4° C.). In some embodiments, a lysed and disrupted sample is further subjected to precipitation of proteins and/or nucleic acids (e.g., using trichloroacetic acid at 4° C. with vortexing) and optionally followed by centrifugation.

In some embodiments, a sample (e.g., a sample comprising a target nucleic acid) may be purified, e.g., following lysis, in a process in accordance with the instant disclosure. In some embodiments, a sample may be purified using chromatography (e.g., affinity chromatography that selectively binds the sample) or electrophoresis. In some embodiments, a sample may be purified in the presence of precipitating agents. In some embodiments, after a purification step or method, a sample may be washed and/or released from a purification matrix (e.g., affinity chromatography matrix) using an elution buffer. In some embodiments, a purification step or method may comprise the use of a reversibly switchable polymer, such as an electroactive polymer. In some embodiments, a sample may be purified by electrophoretic passage of a sample through a porous matrix (e.g., cellulose acetate, agarose, acrylamide).

In some embodiments, a sample (e.g., a sample comprising a target nucleic acid) may be fragmented (i.e., digested) in a process in accordance with the instant disclosure. In some embodiments, a nucleic acid sample may be fragmented to produce small (<1 kilobase) fragments for sequence specific identification to large (up to 10+ kilobases) fragments for long read sequencing applications. Fragmentation of nucleic acids may, in some embodiments, be accomplished using mechanical (e.g., fluidic shearing), chemical (e.g., iron (Fe+) cleavage) and/or enzymatic (e.g., restriction enzymes, tagmentation using transposases) methods. In some embodiments, a nucleic acid may be fragmented by tagmentation such that the nucleic acid is simultaneously fragmented and labeled with a fluorescent molecule (e.g., a fluorophore). In some embodiments, a fragmented sample may be subjected to a round of purification (e.g., chromatography or electrophoresis) to remove small and/or undesired fragments as well as residual payload, chemicals and/or enzymes (e.g., transposases) used during the fragmentation step. For example, a fragmented sample (e.g., sample comprising nucleic acids) may be purified from an enzyme (e.g., a transposase), wherein the purification comprises denaturing the enzyme (e.g., by a combination of heat, chemical (e.g. SDS), and enzymatic (e.g. proteinase K) processes).

In some embodiments, the target molecule(s) is fragmented/digested prior to enrichment. In some embodiments, the target molecule is fragmented/digested after enrichment. In some embodiments, the target molecule(s) is fragmented/digested without any enrichment of the target molecule(s).

Fragmentation/digestion can be conducted using any known method, but typically will involve a non-enzymatic or enzymatic method. Non-enzymatic methods typically have an advantage as it relates to speed, simplicity, robustness, and ease of automation. These approaches include, but are not limited to, acid hydrolysis and/or cleavage using a chemical entity such as cyanogen bromide, hydroxylamine, iodosobenzoic acid, dimethyl sulfoxide-hydrochloric acid, BNPS-skatole [2-(2-nitrophenylsulfenyl)-3-methylindole], or 2-nitro-5-thiocyanobenzoic acid. Non-enzymatic, electro-physical digestion methods have been employed as well, including electrochemical oxidation and/or digestion in conjunction with microwaves.

Enzymatic fragmentation/digestion methods may be optimized for ease of use, speed, automation and/or effectiveness. In some embodiments, enzymatic methods include enzyme immobilization on solid substrates. In some embodiments, enzymatic methods are performed in flow (e.g., in a microfluidic channel).

Fragmentation/digestion methods may be performed using an automated device or module. Alternatively, or in addition, fragmentation/digestion methods may be performed manually. An enzymatic digestion may utilize any number or combination of enzymes and may further comprise any of the known non-enzymatic methods.

In some embodiments, a sample comprising a target nucleic acid may be used to generate a nucleic acid library for subsequent analysis (e.g., genomic sequencing) in a process in accordance with the instant disclosure. A nucleic acid library may be a linear library or a circular library. In some embodiments, nucleic acids of a circular library may comprise elements that allow for downstream linearization (e.g., endonuclease restriction sites, incorporation of uracil). In some embodiments, a nucleic acid library may be purified (e.g., using chromatography, e.g., affinity chromatography, or electrophoresis).

In some embodiments, a library of nucleic acids (e.g., linear nucleic acids) is prepared using end-repair, a process wherein a combination of enzymes (e.g., Taq DNA Ligase, Endonuclease IV, Bst DNA Polymerase, Fpg, Uracil-DNA Glycosylase, T4 Endonuclease V and/or Endonuclease VIII) extend the 3′ end of the nucleic acids, generating a complement to the 5′ payload, and repairing any abasic sites or nicks in the nucleic acids. In some embodiments, a library of linear nucleic acids is prepared using a self-priming hairpin adaptor, a process which may obviate the need to anneal a unique sequencing primer to an individual nucleic acid fragment primer prior to formation of a polymerase complex. Following end-repair, a library of nucleic acids (e.g., linear nucleic acids) may be purified using solid-phase adsorption with subsequent elution into a fresh buffer, using passage of the nucleic acids through a size-selective matrix (e.g., agarose gel). The size-selective matrix may be used to remove nucleic acid fragments that are smaller than the size of the target nucleic acids.

### Application: Library Preparation for Sequencing

The basic application for the platform is the generation of nucleic acid libraries for subsequent genomic sequencing. Utilizing the proper adaptor sequences enables use in any of the commercially available sequencers, but the system's ability to generate long fragments makes it particularly suited to long read sequencing platforms. The system can generate libraries in either a linear, single-stranded format or a closed, circular double-stranded DNA format depending on the requirements for subsequent manipulation.

### Linear Libraries

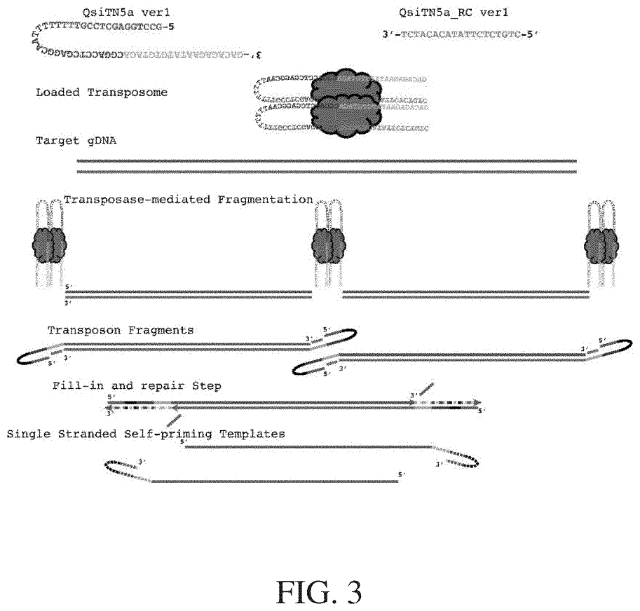

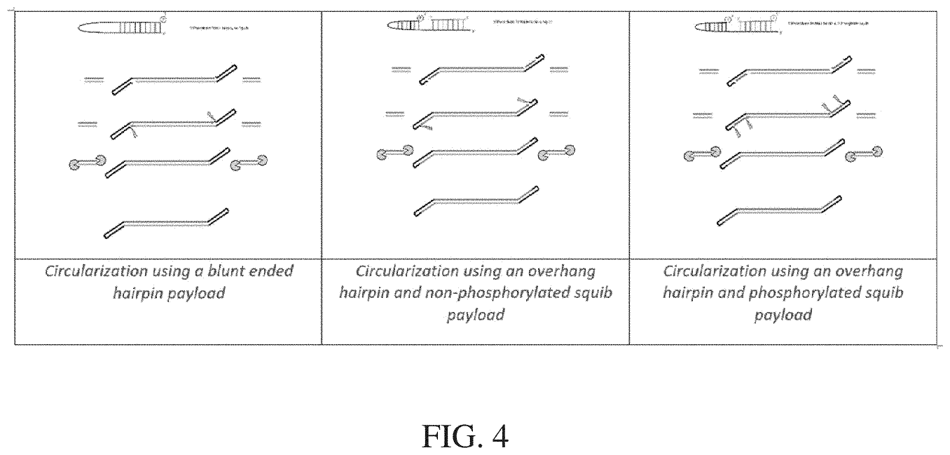

Generation of a library of linear fragments follows a workflow like that outlined in FIG. 3. Following fragmentation, the purified DNA is “end repaired”, wherein a combination of enzymes, such as those provided in the NEBPreCR Repair kit (https://www.neb.com/products/m0309-precr-repair-mix#Product%20Information) (Diegoli, Farr et al. 2012) extend the 3′ end of the fragmented DNA, creating the complement to the 5′ payload, and repair any abasic sites or nicks in the DNA. The cartoon depicts the use of a self-priming hairpin adaptor, obviating the need to anneal a discrete sequencing primer to the fragment primer prior to formation of the polymerase complex, but this is not required and a straight linear fragment with subsequent sequencing primer hybridization could be employed.

Following end repair, the library is purified through either solid-phase adsorption with subsequent elution into a fresh buffer or passage through a size-selective matrix such as an agarose gel. This step can also be combined with a size selection process to remove fragments significantly smaller than the target size. For example, solid phase DNA can be tuned to preferentially select rough size ranges of DNA, and gel-based electrophoresis can select specific size ranges while rejecting nucleotide and protein contaminants. Either process could be supported on the cartridge as dictated by the application needs; solid phase capture is quicker and easier, while gel-based systems are slower but have greater control over size selection.

### Circular Libraries

For some applications it may be preferable to generate libraries of circularized DNA fragments. The benefits of circularization include the ability to treat the sample with exonucleases following ligation, thereby digesting any material which failed to fully circularize. This reduces the amount of material that must be processed in any subsequent size or sequence-specific selection step. Additionally, a circularized template provides the ability to sequence a given insert multiple times as the polymerase circumnavigates the molecule. This interrogates a given base multiple times from both sense and antisense strands, thus improving accuracy. As benefits accrue from multiple reads of the same molecule, these applications typically utilize shorter inserts than those linear long read sequencing.

When generating a circularized product, a different Tn5 payload is employed, wherein the payload contains a 5′ phosphate to enable subsequent ligation. In this case, the enzymatic treatment differs from the previous method in that an initial step of extension uses a non-strand displacing polymerase (e.g., T4 DNA polymerase) to extend the 3′ end of the fragments along the 5′ payload up to the terminal, phosphorylated base of the hairpin. A ligase (e.g., Ampligase) is then used to covalently attach the nascent stretch of extended DNA to the 5′ end of the hairpin, thereby forming a double-stranded closed circular DNA molecule. This process was used by Wang et. Al (Wang, Gu et al. 2013) for gap fill and ligation along a linear adaptor. Standard end repair can then be employed to repair any residual DNA damage. If desired, it is possible to combine some or all the enzymes into a single reaction and accomplish all the modifications in a single step.

It may also be advantageous, however, to initially process the libraries as circular strands (for the exonuclease cleanup) but then linearize strands for subsequent sequencing. This would enable the use of longer fragments, as circumnavigation would not be required. As an example, long linear strands can be utilized as scaffold sequences even at lower per base accuracies. These scaffolds serve to orient high density, shorter reads, increasing the consensus accuracy through oversampling.

A non-comprehensive list of some such moieties includes a rare recognition site for a restriction enzyme in the hairpin, with the corresponding restriction enzyme employed for linearization, incorporation of uracil bases in the hairpin, with UDG (uracil deglycosylase) or other uracil-specific digestion mechanisms utilized for cleavage, or RNA bases incorporated in the hairpin, and RNA-specific digestion (e.g., RNAseH) to linearize the circle. Once linearized, the fragments can be denatured to form two single stranded linear templates, with the free 3′ end of the fragment folding back on itself to form a self-priming hairpin for subsequent sequencing. Alternatively, the sense and antisense strands could remain hybridized, and the sequencing simply initiates from either of the free 3′ fragment ends.

Maximum efficiency for sequencing unknown genomes or complex mixes from metagenomic samples could be achieved by mixing both single and multiple pass sequencing in a single sequencing run. In this case, the library would proceed through generation of a circular library, but with a broad size selection criterion, encompassing the size range desired for shorter multipass circular reads as well that more typical for longer linear reads. When the material is sequenced, the polymerases will cover the shorter templates multiple times while the longer circles will be sequenced fewer times, possibly even once or less. This would provide both single pass, long read scaffolds for genomic orientation, identification of large sale genomic rearrangements, etc., but buttress those reads with the higher accuracy multipass reads.

In some embodiments, a sample (e.g., a sample comprising a target nucleic acid) may be enriched for a target molecule in a process in accordance with the instant disclosure. Enrichment is typically used when the complexity of the un-enriched sample exceeds the capacity of the sequencing platform, or when the target molecule is present in the sample at a low abundance (e.g., such that it cannot be easily detected by the sequencing platform). Enrichment involves the use of a mechanism that selectively amplifies the target molecule. This enrichment may involve the use of antibodies, aptamers, size-based selection, or electrostatic charge-based selection in order to selectively amplify the target molecule(s) (e.g., target nucleic acid(s)).

Enrichment may typically be used when the intent of the sample preparation is to sequence specific target molecules. Enrichment may be used to perform or conduct a proteomic, genomic, or metagenomic analysis or survey, when the target molecules are related or homologous to one another.

In some embodiments, a sample is enriched for a target molecule using an electrophoretic method. In some embodiments, a sample is enriched for a target molecule using affinity SCODA. In some embodiments, a sample is enriched for a target molecule using field inversion gel electrophoresis (FIGE). In some embodiments, a sample is enriched for a target molecule using pulsed field gel electrophoresis (PFGE). In some embodiments, the matrix used during enrichment (e.g., a porous media, electrophoretic polymer gel) comprises immobilized affinity agents (also known as ‘immobilized capture probes’) that bind to target molecule present in the sample. In some embodiments, a matrix used during enrichment comprises 1, 2, 3, 4, 5, or more unique immobilized capture probes, each of which binds to a unique target molecule and/or bind to the same target molecule with different binding affinities.

In some embodiments, an immobilized capture probe is an oligonucleotide capture probe that hybridizes to a target nucleic acid. In some embodiments, an oligonucleotide capture probe is at least 50%, 60%, 70%, 80%, 90% 95%, or 100% complementary to a target nucleic acid. In some embodiments, a single oligonucleotide capture probe may be used to enrich a plurality of related target nucleic acids (e.g., 2, 3, 4, 5, 6, 7, 8, 9, 10, 20, 30, 40, 50, or more related target nucleic acids) that share at least 50%, 60%, 70%, 80%, 90% 95%, or 99% sequence identity. Enrichment of a plurality of related target nucleic acids may allow for the generation of a metagenomic library. In some embodiments, an oligonucleotide capture probe may enable differential enrichment of related target nucleic acids. In some embodiments, an oligonucleotide capture probe may enable enrichment of a target nucleic acid relative to a nucleic acid of identical sequence that differs in its modification state (e.g., single nucleotide polymorphism, methylation state, acetylation state). In some embodiments, an oligonucleotide capture probe is used to enrich human genomic DNA for a specific gene of interest (e.g., HLA). A specific gene of interest may be a gene that is relevant to a specific disease state or disorder. In some embodiments, an oligonucleotide capture probe is used to enrich nucleic acid(s) of a metagenomic sample.

In some embodiments, for the purposes of enriching nucleic acid target molecules with a length of 0.5-2 kilobases, oligonucleotide capture probes may be covalently immobilized in an acrylamide matrix using a 5′ Acrydite moiety. In some embodiments, for the purposes of enriching larger nucleic acid target molecules (e.g., with a length of >2 kilobases), oligonucleotide capture probes may be immobilized in an agarose matrix. In some embodiments, oligonucleotide capture probes may be immobilized in an agarose matrix using thiol-epoxide chemistries (e.g., by covalently attached thiol-modified oligonucleotides to crosslinked agarose beads). Oligonucleotide capture probes linked to agarose beads can be combined and solidified within standard agarose matrices (e.g., at the same agarose percentage).

In some embodiments, enrichment of nucleic acids using methods described herein (e.g., enrichment using SCODA) produces nucleic acid target molecules that comprise a length of about 0.5 kilobases (kb), about 1 kb, about 1.5 kb, about 2 kb, about 3 kb, about 4 kb, about 5 kb, about 6 kb, about 7 kb, about 8 kb, about 9 kb, about 10 kb, about 12 kb, about 15 kb, about 20 kb, or more. In some embodiments, enrichment of nucleic acids using methods described herein (e.g., enrichment using SCODA) produces nucleic acid target molecules that comprise a length of about 0.5-2 kb, 0.5-5 kb, 1-2 kb, 1-3 kb, 1-4 kb, 1-5 kb, 1-10 kb, 2-10 kb, 2-5 kb, 5-10 kb, 5-15 kb, 5-20 kb, 5-25 kb, 10-15 kb, 10-20 kb, or 10-25 kb.

Enrichment of a sample (e.g., a sample comprising a target nucleic acid) allows for a reduction in the total volume of the sample. For example, in some embodiments, the total volume of a sample is reduced after enrichment by at least 10%, at least 20%, at least 30%, at least 40%, at least 50%, at least 60%, at least 70%, at least 80%, at least 90%, at least 100%, or at least 120%. In some embodiments, the total volume of a sample is reduced after enrichment from 1-20 mL initial volume to 100-1000 μL final volume, from 1-5 mL initial volume to 100-1000 μL final volume, from 100-1000 μL initial volume to 25-100 μL final volume, from 100-500 μL initial volume to 10-100 μL final volume, or from 50-200 μL initial volume to 1-25 μL final volume. For example, in some embodiments, the final volume of a sample after enrichment is 10-100 μL, 10-50 μL, 10-25 μL, 20-100 μL, 20-50 μL, 25-100 μL, 25-250 μL, 25-1000 μL, 100-1000 μL, 100-500 μL, 100-250 μL, 200-1000 μL, 200-500 μL, 200-750 μL, 500-1000 μL, 500-1500 μL, 500-750 μL, 1-5 mL, 1-10 mL, 1-2 mL, 1-3 mL, or 1-4 mL.

In addition to amplification of the target molecule, or as an alternative to amplification of the target molecule, a sample may be enriched (e.g., for a low abundance target molecule) by depletion of unwanted non-target molecules (e.g., high-abundance proteins (e.g. albumin)). Depletion of unwanted non-target molecules may be performed using similar capture strategies as discussed above. When using a depletion strategy, the capture probes will bind to unwanted, non-target molecules and allow for target molecules to remain in solution. This strategy equally enables enrichment of the target molecule (i.e., increased relative concentrations of the target molecule(s)).

For example, an immobilized capture probe that is used for depletion may be an oligonucleotide capture probe that hybridizes to an unwanted non-target nucleic acid. In some embodiments, an oligonucleotide capture probe that is used for depletion is at least 50%, 60%, 70%, 80%, 90% 95%, or 100% complementary to an unwanted non-target nucleic acid. In some embodiments, a single oligonucleotide capture probe that is used for depletion may be used to deplete a plurality of related target nucleic acids (e.g., 2, 3, 4, 5, 6, 7, 8, 9, 10, 20, 30, 40, 50, or more related target nucleic acids) that share at least 50%, 60%, 70%, 80%, 90% 95%, or 99% sequence identity.

In some embodiments, any number of enrichment steps (e.g., amplification of target molecule(s) and/or depletion(s)) can be performed by the automated device or module (e.g., on a chip or cartridge). In some embodiments, the enrichment steps are amenable to automation on the cartridge using capture elements (e.g., antibodies) immobilized on solid phase structures. In some embodiments, any immobilized capture element or probe described herein may be on any solid support structure or surface. The solid support structure or surface may be magnetic and/or may be a frit, a filter, a chip, or a cartridge surface. In some embodiments, the capture elements or probes for enrichment may be interchanged (e.g., using flow on a chip). In some embodiments, any number of the enrichment steps are performed manually. If performed manually, any enriched target molecule may be subsequently placed into an automated sample preparation device described herein.

In some embodiments, a target molecule or target molecules may be detected after enrichment and subsequent release to enable analysis of said target molecule(s) and its upstream sample, in a process in accordance with the instant disclosure. In some embodiments, a target nucleic acid may be detected using gene sequencing, absorbance, fluorescence, electrical conductivity, capacitance, surface plasmon resonance, hybrid capture, antibodies, direct labeling of the nucleic acid (e.g., end-labeling, labeled tagmentation payloads), non-specific labeling with intercalating dyes (e.g., ethidium bromide, SYBR dyes), or any other known methodology for nucleic acid detection.

Devices or modules including apparatuses, cartridges (e.g., comprising channels (e.g., microfluidic channels)), and/or pumps (e.g., peristaltic pumps) for use in a process of preparing a sample for analysis are generally provided. Devices can be used in accordance with the instant disclosure to promote capture, concentration, manipulation, and/or detection of a target molecule from a biological sample. In some embodiments, devices and related methods are provided for automated processing of a sample to produce material for next generation sequencing and/or other downstream analytical techniques. Devices and related methods may be used for performing chemical and/or biological reactions, including reactions for nucleic acid processing in accordance with sample preparation or sample analysis processes described elsewhere herein.

In some embodiments, a cartridge comprises one or more reservoirs or reaction vessels configured to receive a fluid and/or contain one or more reagents used in a sample preparation process. In some embodiments, a cartridge comprises one or more channels (e.g., microfluidic channels) configured to contain and/or transport a fluid (e.g., a fluid comprising one or more reagents) used in a sample preparation process. Reagents include buffers, enzymatic reagents, polymer matrices, capture reagents, size-specific selection reagents, sequence-specific selection reagents, and/or purification reagents. Additional reagents for use in a sample preparation process are described elsewhere herein.

In some embodiments, a cartridge includes one or more stored reagents (e.g., of a liquid or lyophilized form suitable for reconstitution to a liquid form). The stored reagents of a cartridge include reagents suitable for carrying out a desired process and/or reagents suitable for processing a desired sample type. In some embodiments, a cartridge is a single-use cartridge (e.g., a disposable cartridge) or a multiple-use cartridge (e.g., a reusable cartridge). In some embodiments, a cartridge is configured to receive a user-supplied sample. The user-supplied sample may be added to the cartridge before or after the cartridge is received by the device, e.g., manually by the user or in an automated process. In some embodiments, a cartridge is a sample preparation cartridge. In some embodiments, a sample preparation cartridge is capable of isolating or purifying a target molecule (e.g., a target nucleic acid) from a sample (e.g., a biological sample).

In some embodiments, a cartridge comprises an affinity matrix for enrichment as described herein. In some embodiments, a cartridge comprises an affinity matrix for enrichment using affinity SCODA, FIGE, or PFGE. In some embodiments, a cartridge comprises an affinity matrix comprising an immobilized affinity agent that has a binding affinity for a target nucleic acid.

In some embodiments, a sample preparation device of the disclosure produces (e.g., enriches or purifies) target nucleic acids with an average read-length for downstream sequencing applications that is longer than an average read-length produced using control methods (e.g., Sage BluePippin methods, manual methods (e.g., manual bead-based size selection methods)). In some embodiments, a sample preparation device produces target nucleic acids with an average read-length for sequencing that comprises at least 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides in length. In some embodiments, a sample preparation device produces target nucleic acids with an average read-length for sequencing that comprises 700-3000, 1000-3000, 1000-2500, 1000-2400, 1000-2300, 1000-2200, 1000-2100, 1000-2000, 1000-1900, 1000-1800, 1000-1700, 1000-1600, 1000-1500, 1000-1400, 1000-1300, 1000-1200, 1500-3000, 1500-2500, 1500-2000, or 2000-3000 nucleotides in length.

Devices in accordance with the instant disclosure generally contain mechanical and electronic and/or optical components which can be used to operate a cartridge as described herein. In some embodiments, the device components operate to achieve and maintain specific temperatures on a cartridge or on specific regions of the cartridge. In some embodiments, the device components operate to apply specific voltages for specific time durations to electrodes of a cartridge. In some embodiments, the device components operate to move liquids to, from, or between reservoirs and/or reaction vessels of a cartridge. In some embodiments, the device components operate to move liquids through channel(s) of a cartridge, e.g., to, from, or between reservoirs and/or reaction vessels of a cartridge. In some embodiments, the device components move liquids via a peristaltic pumping mechanism (e.g., apparatus) that interacts with an elastomeric, reagent-specific reservoir or reaction vessel of a cartridge. In some embodiments, the device components move liquids via a peristaltic pumping mechanism (e.g., apparatus) that is configured to interact with an elastomeric component (e.g., surface layer comprising an elastomer) associated with a channel of a cartridge to pump fluid through the channel. Device components can include computer resources, for example, to drive a user interface where sample information can be entered, specific processes can be selected, and run results can be reported.

In some embodiments, a cartridge is capable of handling small-volume fluids (e.g., 1-10 μL, 2-10 μL, 4-10 μL, 5-10 μL, 1-8 μL, or 1-6 μL fluid). In some embodiments, the sequencing cartridge is physically embedded or associated with a sample preparation device or module (e.g., to allow for a prepared sample to be delivered to a reaction mixture for sequencing. In some embodiments, a sequencing cartridge that is physically embedded or associated with a sample preparation device or module comprises microfluidic channels that have fluid interfaces in the form of face sealing gaskets or conical press fits (e.g., Luer fittings). In some embodiments, fluid interfaces can then be broken after delivery of the prepared sample in order to physically separate the sequencing cartridge from the sample preparation device or module.

The following non-limiting example is meant to illustrate aspects of the devices, methods, and compositions described herein. The use of a sample preparation device or module in accordance with the instant disclosure may proceed with one or more of the following described steps. A user may open the lid of the device and insert a cartridge that supports the desired process. The user may then add a sample, which may be combined with a specific lysis solution, to a sample port on the cartridge. The user may then close the device lid, enter any sample specific information via a touch screen interface on the device, select any process specific parameters (e.g., range of desired size selection, desired degree of homology for target molecule capture, etc.), and initiate the sample preparation process run. Following the run, the user may receive relevant run data (e.g., confirmation of successful completion of the run, run specific metrics, etc.), as well as process specific information (e.g., amount of sample generated, presence or absence of specific target sequence, etc.). Data generated by the run may be subjected to subsequent bioinformatics analysis, which can be either local or cloud based. Depending on the process, a finished sample may be extracted from the cartridge for subsequent use (e.g., genomic sequencing, qPCR quantification, cloning, etc.). The device may then be opened, and the cartridge may then be removed.

In some embodiments, the sample preparation module comprises a pump. In some embodiments, the pump is peristaltic pump. Some such pumps comprise one or more of the inventive components for fluid handling described herein. For example, the pump may comprise an apparatus and/or a cartridge. In some embodiments, the apparatus of the pump comprises a roller, a crank, and a rocker. In some such embodiments, the crank and the rocker are configured as a crank-and-rocker mechanism that is connected to the roller. The coupling of a crank-and-rocker mechanism with the roller of an apparatus can, in some cases, allow for certain of the advantages describe herein to be achieved (e.g., facile disengagement of the apparatus from the cartridge, well-metered stroke volumes). In certain embodiments, the cartridge of the pump comprises channels (e.g., microfluidic channels). In some embodiments, at least a portion of the channels of the cartridge have certain cross-sectional shapes and/or surface layers that may contribute to any of a number of advantages described herein.

One non-limiting aspect of some cartridges that may, in some cases, provide certain benefits is the inclusion of channels having certain cross-sectional shapes in the cartridges. For example, in some embodiments, the cartridge comprises v-shaped channels. One potentially convenient but non-limiting way to form such v-shaped channels is by molding or machining v-shaped grooves into the cartridge. The recognized advantages of including a v-shaped channel (also referred to herein as a v-groove or a channel having a substantially triangularly-shaped cross-section) in certain embodiments in which a roller of the apparatus engages with the cartridge to cause fluid flow through the channels. For example, in some instances, a v-shaped channel is dimensionally insensitive to the roller. In other words, in some instances, there is no single dimension to which the roller (e.g., a wedge shaped roller) of the apparatus must adhere in order to suitably engage with the v-shaped channel. In contrast, certain conventional cross sectional shapes of the channels, such as semi-circular, may require that the roller have a certain dimension (e.g., radius) in order to suitably engage with the channel (e.g., to create a fluidic seal to cause a pressure differential in a peristaltic pumping process). In some embodiments, the inclusion of channels that are dimensionally insensitive to rollers can result in simpler and less expensive fabrication of hardware components and increased configurability/flexibility.

In certain aspects, the cartridges comprise a surface layer (e.g., a flat surface layer). One exemplary aspect relates to potentially advantageous embodiments involving layering a membrane (also referred to herein as a surface layer) comprising (e.g., consisting essentially of) an elastomer (e.g., silicone) above the v-groove, to produce, in effect, half of a flexible tube. Then, in some embodiments, by deforming the surface layer comprising an elastomer into the channel to form a pinch and by then translating the pinch, negative pressure can be generated on the trailing edge of the pinch which creates suction and positive pressure can be generated on the leading edge of the pinch, pumping fluid in the direction of the leading edge of the pinch. In certain embodiments, this pumping by interfacing a cartridge (comprising channels having a surface layer) with an apparatus comprising a roller, which apparatus is configured to carry out a motion of the roller that includes engaging the roller with a portion of the surface layer to pinch the portion of the surface layer with the walls and/or base of the associated channel, translating the roller along the walls and/or base of the associated channel in a rolling motion to translate the pinch of the surface layer against the walls and/or base, and/or disengaging the roller with a second portion of the surface layer. In certain embodiments, a crank-and-rocker mechanism is incorporated into the apparatus to carry out this motion of the roller.

A conventional peristaltic pump generally involves tubing having been inserted into an apparatus comprising rollers on a rotating carriage, such that the tubing is always engaged with the remainder of the apparatus as the pump functions. By contrast, in certain embodiments, channels in cartridges herein are linear or comprise at least one linear portion, such that the roller engages with a horizontal surface. In certain embodiments, the roller is connected to a small roller arm that is spring-loaded so that the roller can track the horizontal surface while continuously pinching a portion of the surface layer. Spring loading the apparatus (e.g., a roller arm of the apparatus) can in some cases help regulate the force applied by the apparatus (e.g., roller) to the surface layer and a channel of a cartridge.

In certain embodiments, each rotation of the crank in a crank-and-rocker mechanism connected to the roller provides a discrete pumping volume. In certain embodiments, it is straightforward to park the apparatus in a disengaged position, where the roller is disengaged from any cartridge. In certain embodiments, forward and backward pumping motions are fairly symmetrical as provided by apparatuses described herein, such that a similar amount of force (torque) (e.g., within 10%) is required for forward and backward pumping motions.

In certain embodiments, it may be advantageous to, for a particular size of apparatus, have a relatively high crank radius (e.g., greater than or equal to 2 mm, optionally including associated linkages). Consequently, it may, in certain embodiments, also be advantageous to have a relatively high stroke length (e.g., greater than or equal to 10 mm) to engage with an associated cartridge. Having relatively high crank radius and stroke length, in certain embodiments, ensures no mechanical interference between the apparatus and the cartridge when moving components of the apparatus relative to the cartridge.

In certain embodiments, having v-shaped grooves advantageously allows for utilization with rollers of a variety of sizes having a wedge-shaped edge. By contrast, for example, having a rectangular channel rather than a v-groove results in the width of the roller associated with the rectangular channel needing to be more controlled and precise in relation to the width of the rectangular channel, and results in the forces being applied to the rectangular channel needing to be more precise. Similarly, the channel(s) having a semicircular cross-section may also require more controlled and precise dimension for the width of the associated roller.

In certain embodiments, an apparatus described herein may comprise a multi-axis system (e.g., robot) configured so as to move at least a portion of the apparatus in a plurality of dimensions (e.g., two dimensions, three dimensions). For example, the multi-axis system may be configured so as to move at least a portion of the apparatus to any pumping lane location among associated cartridge(s). For example, in certain embodiments, a carriage herein may be functionally connected to a multi-axis system. In certain embodiments, a roller may be indirectly functionally connected to a multi-axis system. In certain embodiments, an apparatus portion, comprising a crank-and-rocker mechanism connected to a roller, may be functionally connected to a multi-axis system. In certain embodiments, each pumping lane may be addressed by location and accessed by an apparatus described herein using a multi-axis system.

### Nucleic Acid Sequencing Process

Some aspects of the instant disclosure further involve sequencing nucleic acids (e.g., deoxyribonucleic acids or ribonucleic acid). In some aspects, compositions, devices, systems, and techniques described herein can be used to identify a series of nucleotides incorporated into a nucleic acid (e.g., by detecting a time-course of incorporation of a series of labeled nucleotides). In some embodiments, compositions, devices, systems, and techniques described herein can be used to identify a series of nucleotides that are incorporated into a template-dependent nucleic acid sequencing reaction product synthesized by a polymerizing enzyme (e.g., RNA polymerase).

Accordingly, also provided herein are methods of determining the sequence of a target nucleic acid. In some embodiments, the target nucleic acid is enriched (e.g., enriched using electrophoretic methods, e.g., affinity SCODA) prior to determining the sequence of the target nucleic acid. In some embodiments, provided herein are methods of determining the sequences of a plurality of target nucleic acids (e.g., at least 2, 3, 4, 5, 10, 15, 20, 30, 50, or more) present in a sample (e.g., a purified sample, a cell lysate, a single-cell, a population of cells, or a tissue). In some embodiments, a sample is prepared as described herein (e.g., lysed, purified, fragmented, and/or enriched for a target nucleic acid) prior to determining the sequence of a target nucleic acid or a plurality of target nucleic acids present in a sample. In some embodiments, a target nucleic acid is an enriched target nucleic acid (e.g., enriched using electrophoretic methods, e.g., affinity SCODA).

In some embodiments, methods of sequencing comprise steps of: (i) exposing a complex in a target volume to one or more labeled nucleotides, the complex comprising a target nucleic acid or a plurality of nucleic acids present in a sample, at least one primer, and a polymerizing enzyme; (ii) directing one or more excitation energies, or a series of pulses of one or more excitation energies, towards a vicinity of the target volume; (iii) detecting a plurality of emitted photons from the one or more labeled nucleotides during sequential incorporation into a nucleic acid comprising one of the at least one primers; and (iv) identifying the sequence of incorporated nucleotides by determining one or more characteristics of the emitted photons.

In another aspect, the instant disclosure provides methods of sequencing target nucleic acids or a plurality of target nucleic acids present in a sample by sequencing a plurality of nucleic acid fragments, wherein the target nucleic acid(s) comprises the fragments. In certain embodiments, the method comprises combining a plurality of fragment sequences to provide a sequence or partial sequence for the parent nucleic acid (e.g., parent target nucleic acid). In some embodiments, the step of combining is performed by computer hardware and software. The methods described herein may allow for a set of related nucleic acids (e.g., two or more nucleic acids present in a sample), such as an entire chromosome or genome to be sequenced. In some embodiments, a primer is a sequencing primer. In some embodiments, a sequencing primer can be annealed to a nucleic acid (e.g., a target nucleic acid) that may or may not be immobilized to a solid support. A solid support can comprise, for example, a sample well (e.g., a nanoaperture, a reaction chamber) on a chip or cartridge used for nucleic acid sequencing. In some embodiments, a sequencing primer may be immobilized to a solid support and hybridization of the nucleic acid (e.g., the target nucleic acid) further immobilizes the nucleic acid molecule to the solid support. In some embodiments, a polymerase (e.g., RNA Polymerase) is immobilized to a solid support and soluble sequencing primer and nucleic acid are contacted to the polymerase. In some embodiments a complex comprising a polymerase, a nucleic acid (e.g., a target nucleic acid) and a primer is formed in solution and the complex is immobilized to a solid support (e.g., via immobilization of the polymerase, primer, and/or target nucleic acid). In some embodiments, none of the components are immobilized to a solid support. For example, in some embodiments, a complex comprising a polymerase, a target nucleic acid, and a sequencing primer is formed in situ and the complex is not immobilized to a solid support. In some embodiments, sequencing by synthesis methods can include the presence of a population of target nucleic acid molecules (e.g., copies of a target nucleic acid) and/or a step of amplification (e.g., polymerase chain reaction (PCR)) of a target nucleic acid to achieve a population of target nucleic acids. However, in some embodiments, sequencing by synthesis is used to determine the sequence of a single nucleic acid molecule in any one reaction that is being evaluated and nucleic acid amplification may not be required to prepare the target nucleic acid. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells (e.g., nanoapertures, reaction chambers) on a single chip or cartridge.

In some embodiments, sequencing of a target nucleic acid molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) nucleotides of the target nucleic acid. In some embodiments, the at least two nucleotides are contiguous nucleotides. In some embodiments, the at least two amino acids are non-contiguous nucleotides. In some embodiments, sequencing of a target nucleic acid comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all nucleotides in the target nucleic acid. For example, in some embodiments, sequencing of a target nucleic acid comprises identification of less than 100% of one type of nucleotide in the target nucleic acid. In some embodiments, sequencing of a target nucleic acid comprises identification of less than 100% of each type of nucleotide in the target nucleic acid.

### Sequencing Device or Module

Sequencing of nucleic acids in accordance with the instant disclosure, in some aspects, may be performed using a system that permits single molecule analysis. The system may include a sequencing device or module and an instrument configured to interface with the sequencing device or module. The sequencing device or module may include an array of pixels, where individual pixels include a sample well and at least one photodetector. The sample wells of the sequencing device or module may be formed on or through a surface of the sequencing device or module and be configured to receive a sample placed on the surface of the sequencing device or module. In some embodiments, the sample wells are a component of a cartridge (e.g., a disposable or single-use cartridge) that can be inserted into the device. Collectively, the sample wells may be considered as an array of sample wells. The plurality of sample wells may have a suitable size and shape such that at least a portion of the sample wells receive a single target molecule or sample comprising a plurality of molecules (e.g., a target nucleic acid). In some embodiments, the number of molecules within a sample well may be distributed among the sample wells of the sequencing device or module such that some sample wells contain one molecule (e.g., a target nucleic acid) while others contain zero, two, or a plurality of molecules.

In some embodiments, a sequencing device or module is positioned to receive a target molecule or sample comprising a plurality of molecules (e.g., a target nucleic acid) from a sample preparation device or module. In some embodiments, a sequencing device or module is connected directly (e.g., physically attached to) or indirectly to a sample preparation device or module.

Excitation light is provided to the sequencing device or module from one or more light sources external to the sequencing device or module. Optical components of the sequencing device or module may receive the excitation light from the light source and direct the light towards the array of sample wells of the sequencing device or module and illuminate an illumination region within the sample well. In some embodiments, a sample well may have a configuration that allows for the target molecule or sample comprising a plurality of molecules to be retained in proximity to a surface of the sample well, which may ease delivery of excitation light to the sample well and detection of emission light from the target molecule or sample comprising a plurality of molecules. A target molecule or sample comprising a plurality of molecules positioned within the illumination region may emit emission light in response to being illuminated by the excitation light. For example, a nucleic acid (or pluralities thereof) may be labeled with a fluorescent marker, which emits light in response to achieving an excited state through the illumination of excitation light. Emission light emitted by a target molecule or sample comprising a plurality of molecules may then be detected by one or more photodetectors within a pixel corresponding to the sample well with the target molecule or sample comprising a plurality of molecules being analyzed. When performed across the array of sample wells, which may range in number between approximately 10,000 pixels to 1,000,000 pixels according to some embodiments, multiple sample wells can be analyzed in parallel.

The sequencing device or module may include an optical system for receiving excitation light and directing the excitation light among the sample well array. The optical system may include one or more grating couplers configured to couple excitation light to the sequencing device or module and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the sequencing device or module by improving the uniformity of excitation light received by sample wells of the sequencing device or module. Examples of suitable components, e.g., for coupling excitation light to a sample well and/or directing emission light to a photodetector, to include in a sequencing device or module are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated herein by reference in their entirety. Examples of suitable grating couplers and waveguides that may be implemented in the sequencing device or module are described in U.S. patent application Ser. No. 15/844,403, filed Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the sequencing device or module, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated herein by reference in its entirety.

Components located off of the sequencing device or module may be used to position and align an excitation source to the sequencing device or module. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated herein by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference in its entirety. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the sequencing device or module may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device or module, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device or module, which may be connected to an instrument interfaced with the sequencing device or module. The electrical signals may be subsequently processed and/or analyzed. Processing and/or analyzing of electrical signals may occur on a suitable computing device either located on or off the instrument.

The instrument may include a user interface for controlling operation of the instrument and/or the sequencing device or module. The user interface may be configured to allow a user to input information into the instrument, such as commands and/or settings used to control the functioning of the instrument. In some embodiments, the user interface may include buttons, switches, dials, and/or a microphone for voice commands. The user interface may allow a user to receive feedback on the performance of the instrument and/or sequencing device or module, such as proper alignment and/or information obtained by readout signals from the photodetectors on the sequencing device or module. In some embodiments, the user interface may provide feedback using a speaker to provide audible feedback. In some embodiments, the user interface may include indicator lights and/or a display screen for providing visual feedback to a user.

In some embodiments, the instrument or device described herein may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device or module, and/or data generated from the readout signals of the photodetector.

In some embodiments, the instrument may include a processing device configured to analyze data received from one or more photodetectors of the sequencing device or module and/or transmit control signals to the excitation source(s). In some embodiments, the processing device may comprise a general purpose processor, and/or a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the sequencing device or module.

According to some embodiments, the instrument that is configured to analyze target molecules or samples comprising a plurality of molecules based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region may be less complex to operate and maintain, may be more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference in its entirety.

In some embodiments, different numbers of fluorophores of the same type may be linked to different components of a target molecule (e.g., a target nucleic acid) or a plurality of molecules present in a sample (e.g., a plurality of nucleic acids), so that each individual molecule may be identified based on luminescence intensity. For example, two fluorophores may be linked to a first labeled molecule and four or more fluorophores may be linked to a second labeled molecule. Because of the different numbers of fluorophores, there may be different excitation and fluorophore emission probabilities associated with the different molecule. For example, there may be more emission events for the second labeled molecule during a signal accumulation interval, so that the apparent intensity of the bins is significantly higher than for the first labeled molecule.

The inventors have recognized and appreciated that distinguishing nucleic acids based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation. For example, a limited bandwidth of radiation may include a central or peak wavelength within a 20 nm bandwidth output by a pulsed optical source. In some cases, “characteristic wavelength” or “wavelength” may be used to refer to a peak wavelength within a total bandwidth of radiation output by a source.

### Combined Sample Preparation and Sequencing Device

In some embodiments, a device herein comprising a sample preparation module further comprises a sequencing module. In some embodiments, a device that comprises a sample preparation module and a sequencing module involves a sequencing chip or cartridge that is embedded into a sample preparation cartridge, such that the two cartridges comprise a single, inseparable consumable. In some embodiments, the sequencing chip or cartridge requires consumable support electronics (e.g., a PCB substrate with wirebonds, electrical contacts). The consumable support electronics may be in direct physical contact with the sequencing chip or cartridge. In some embodiments, the sequencing chip or cartridge requires an interface for a peristaltic pump, temperature control and/or electrophoresis contacts. These interfaces may allow for precise geometric registration for the many electrical contacts and laser alignment. In some embodiments, different sections of a chip or cartridge may comprise different temperatures, physical forces, electrical interfaces of varying voltage and current, vibration, and/or competing alignment requirements. In some embodiments, disparate instrument sub-systems associated with either the sample preparation or sequencing module must be in close proximity in order to share resources. In some embodiments, a device that comprises a sample preparation module and a sequencing module is hands-free (i.e., can be used without the use of hands).

In some embodiments, a device that comprises a sample preparation module and a sequencing module produces (e.g., enriches or purifies) target nucleic acids with an average read-length for downstream sequencing applications that is longer than an average read-length produced using control methods (e.g., Sage BluePippin methods, manual methods (e.g., manual bead-based size selection methods)). In some embodiments, a sample preparation device produces target nucleic acids with an average read-length for sequencing that comprises at least 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides in length. In some embodiments, a sample preparation device produces target nucleic acids with an average read-length for sequencing that comprises 700-3000, 1000-3000, 1000-2500, 1000-2400, 1000-2300, 1000-2200, 1000-2100, 1000-2000, 1000-1900, 1000-1800, 1000-1700, 1000-1600, 1000-1500, 1000-1400, 1000-1300, 1000-1200, 1500-3000, 1500-2500, 1500-2000, or 2000-3000 nucleotides in length.

In some embodiments, a device that comprises a sample preparation module and a sequencing module allows for shortened times between initiation of sample preparation and detection of a target molecule contained within the sample than control or traditional methods (e.g., Sage BluePippin methods followed by sequencing). In some embodiments, a device that comprises a sample preparation module and a sequencing module is capable of detecting a target molecule using sequencing in less time (e.g., 2-fold, 3-fold, 4-fold, 5-fold, or 10-fold less time) than control or traditional methods (e.g., Sage BluePippin methods followed by sequencing).

In some embodiments, a device that comprises a sample preparation module and a sequencing module is capable of detecting a target molecule with lower inputs of sample than control or traditional methods (e.g., Sage BluePippin methods followed by sequencing). In some embodiments, a device of the disclosure requires as little as 0.1 μg, 0.2 μg, 0.3 μg, 0.4 μg, 0.5 μg, 0.6 μg, 0.7 μg, 0.8 μg, 0.9 μg, or 1 μg of sample (e.g., biological sample). In some embodiments, a device of the disclosure requires as little as 10 μL, 20 μL, 30 μL, 40 μL, 50 μL, 60 μL, 70 μL, 80 μL, 90 μL, 100 μL, 110 μL, 130 μL, 150 μL, 175 μL, 200 μL, 225 μL, or 250 μL of sample (e.g., biological sample such as blood).

### Devices or Modules

In some embodiments, devices or modules (e.g., sample preparation devices; sequencing devices; combined sample preparation and sequencing devices) are configured to transport small volume(s) of fluid precisely with a well-defined fluid flow resolution, and with a well-defined flow rate in some cases. In some embodiments, devices or modules are configured to transport fluid at a flow rate of greater than or equal to 0.1 μL/s, greater than or equal to 0.5 μL/s, greater than or equal to 1 μL/s, greater than or equal to 2 μL/s, greater than or equal to 5 μL/s, or higher. In some embodiments, devices or modules herein are configured to transport fluid at a flow rate of less than or equal to 100 μL/s, less than or equal to 75 μL/s, less than or equal to 50 μL/s, less than or equal to 30 μL/s, less than or equal to 20 μL/s, less than or equal to 15 μL/s, or less. Combinations of these ranges are possible. For example, in some embodiments, devices or modules herein are configured to transport fluid at a flow rate of greater than or equal to 0.1 μL/s and less than or equal to 100 μL/s, or greater than or equal to 5 μL/s and less than or equal to 15 μL/s. For example, in certain embodiments, systems, devices, and modules herein have a fluid flow resolution on the order of tens of microliters or hundreds of microliters. Further description of fluid flow resolution is described elsewhere herein. In certain embodiments, systems, devices, and modules are configured to transport small volumes of fluid through at least a portion of a cartridge.

Some aspects relate to configurations of pumps and apparatuses that include a roller (e.g., in combination with a crank-and-rocker mechanism). Other aspects relate to cartridges comprising channels (e.g., microchannels) having cross-sectional shapes (e.g., substantially triangular shapes), valving, deep sections, and/or surface layers (e.g., flat elastomer membranes). Certain aspects relate to a decoupling of certain components of the peristaltic pump (e.g., the roller) from other components of the pump (e.g., pumping lanes). In some cases, certain elements of apparatuses (e.g., edges of the roller) are configured to interact with elements of the cartridge (e.g., surface layers and certain shapes of the channels) in such a way (e.g., via engagement and disengagement) that any of a variety of advantages are achieved. In some non-limiting embodiments, certain inventive features and configurations of the apparatuses, cartridges, and pumps described herein contribute to improved automation of the fluid pumping process (e.g., due to the use of a translatable roller and a separate cartridge containing multiple different fluidic channels that can be indexed by the roller). In some cases, features described herein contribute to an ability to handle a relatively high number of different fluids (e.g., for multiplexing with multiple samples) with a relatively high number of configurations using a relatively small number of hardware components (e.g., due to the use of separate cartridges with multiple different channels, each of which may be accessible to the roller). As one example, in some cases, the features described herein allow for more than one apparatus to be paired with a cartridge to pump more than one lane simultaneously or use two pumps in one lane for other functionality. In some cases, the features contribute to a reduction in required fluid volume and/or less stringent tolerances in roller/channel interactions (e.g., due to inventive cross-sectional shapes of the channels and/or the edge of the roller, and/or due to the use of inventive valving and/or deep sections of channels). In some cases, features described herein result in a reduction in required washing of hardware components (e.g., due to a decoupling of an apparatus and a cartridge of the peristaltic pump). In some embodiments, aspects of the apparatuses, cartridges, and pumps described herein are useful for preparing samples. For example, some such aspects may be incorporated into a sample preparation module upstream of a detection module (e.g., for analysis/sequencing/identification of biologically-derived samples).

In another aspect, peristaltic pumps are provided. In some embodiments, a peristaltic pump comprises a roller and a cartridge, wherein the cartridge comprises a base layer having a surface comprising channels, wherein at least a portion of at least some of the channels (1) have a substantially triangularly-shaped cross-section having a single vertex at a base of the channel and having two other vertices at the surface of the base layer, and (2) have a surface layer, comprising an elastomer, configured to substantially seal off a surface opening of the channel. Embodiments of peristaltic pumps are further described elsewhere herein.

In some embodiments, a system (e.g., pump, device) described herein undergoes a pump cycle. In some embodiments, a pump cycle corresponds to one rotation of a crank of the system. In some embodiments, each pump cycle may transport greater than or equal to 1 μL, greater than or equal to 2 μL, greater than or equal to 4 μL, less than or equal to 10 μL, less than or equal to 8 μL, and/or less than or equal to 6 μL of fluid. Combinations of the above-referenced ranges are also possible (e.g., between or equal to 1 μL and 10 μL). Other ranges of volumes of fluid are also possible.

In some embodiments, a system described herein has a particular stroke length. In certain embodiments, given that each pump cycle may transport on the order of between or equal to 1 μL and 10 μL of fluid, and/or given that channel dimensions may preferably be on the order of 1 mm wide and on the order of 1 mm deep (e.g., depending on what can be machined or molded to decrease channel volume and maintain reasonable tolerances), a stroke length may be greater than or equal to 10 mm, greater than or equal to 12 mm, greater than or equal to 14 mm, less than or equal to 20 mm, less than or equal to 18 mm, and/or less than or equal to 16 mm. Combinations of the above-referenced ranges are also possible (e.g., between or equal to 10 mm and 20 mm). Other ranges are also possible. As used herein, “stroke length” refers to a distance a roller travels while engaged with a substrate. In certain embodiments, the substrate comprises a cartridge.

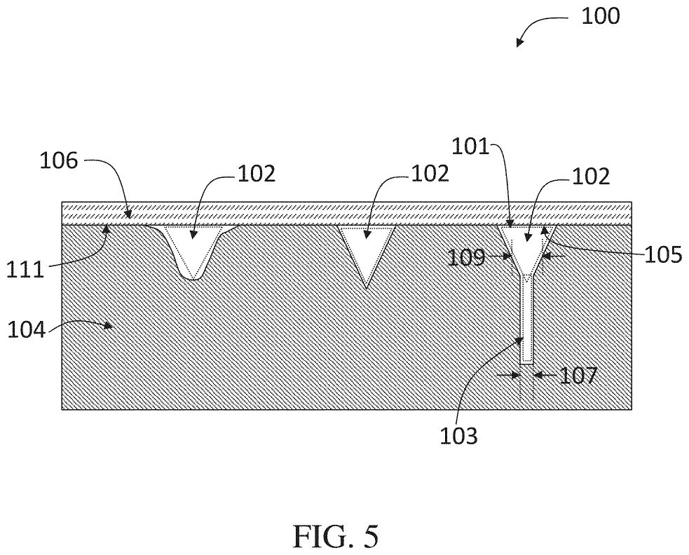

In another aspect, cartridges are provided. In some embodiments, a cartridge comprises a base layer having a surface comprising channels, and at least a portion of at least some of the channels (1) have a substantially triangularly-shaped cross-section having a single vertex at a base of the channel and having two other vertices at the surface of the base layer, and (2) have a surface layer, comprising an elastomer, configured to substantially seal off a surface opening of the channel. Embodiments of cartridges are further described elsewhere herein. In some embodiments, a cartridge comprises a base layer. In some embodiments, a base layer has a surface comprising one or more channels. For example, FIG. 5 is a schematic diagram of a cross-section view of a cartridge 100 along the width of channels 102, in accordance with some embodiments. The depicted cartridge 100 includes a base layer 104 having a surface 111 comprising channels 102. In certain embodiments, at least some of the channels are microchannels. For example, in some embodiments, at least some of channels 102 are microchannels. In certain embodiments, all of the channels microchannels. For example, referring again to FIG. 5, in certain embodiments, all of channels 102 are microchannels. As used herein, the term “channel” will be known to those of ordinary skill in the art and may refer to a structure configured to contain and/or transport a fluid. A channel generally comprises: walls; a base (e.g., a base connected to the walls and/or formed from the walls); and a surface opening that may be open, covered, and/or sealed off at one or more portions of the channel.

As used herein, the term “microchannel” refers to a channel that comprises at least one dimension less than or equal to 1000 microns in size. For example, a microchannel may comprise at least one dimension (e.g., a width, a height) less than or equal to 1000 microns (e.g., less than or equal to 100 microns, less than or equal to 10 microns, less than or equal to 5 microns) in size. In some embodiments, a microchannel comprises at least one dimension greater than or equal to 1 micron (e.g., greater than or equal to 2 microns, greater than or equal to 10 microns). Combinations of the above-referenced ranges are also possible (e.g., greater than or equal to 1 micron and less than or equal to 1000 microns, greater than or equal to 10 micron and less than or equal to 100 microns). Other ranges are also possible. In some embodiments, a microchannel has a hydraulic diameter of less than or equal to 1000 microns. As used herein, the term “hydraulic diameter” (DH) will be known to those of ordinary skill in the art and may be determined as: DH=4A/P, wherein A is a cross-sectional area of the flow of fluid through the channel and P is a wetted perimeter of the cross-section (a perimeter of the cross-section of the channel contacted by the fluid).

In some embodiments, at least a portion of at least some channel(s) have a substantially triangularly-shaped cross-section. In some embodiments, at least a portion of at least some channel(s) have a substantially triangularly-shaped cross-section having a single vertex at a base of the channel and having two other vertices at the surface of the base layer. Referring again to FIG. 5, in some embodiments, at least a portion of at least some of channels 102 have a substantially triangularly-shaped cross-section having a single vertex at a base of the channel and having two other vertices at the surface of the base layer.

As used herein, the term “triangular” is used to refer to a shape in which a triangle can be inscribed or circumscribed to approximate or equal the actual shape, and is not constrained purely to a triangle. For example, a triangular cross-section may comprise a non-zero curvature at one or more portions.

A triangular cross-section may comprise a wedge shape. As used herein, the term “wedge shape” will be known by those of ordinary skill in the art and refers to a shape having a thick end and tapering to a thin end. In some embodiments, a wedge shape has an axis of symmetry from the thick end to the thin end. For example, a wedge shape may have a thick end (e.g., surface opening of a channel) and taper to a thin end (e.g., base of a channel), and may have an axis of symmetry from the thick end to the thin end.

Additionally, in certain embodiments, substantially triangular cross-sections (i.e., “v-groove(s)”) may have a variety of aspect ratios. As used herein, the term “aspect ratio” for a v-groove refers to a height-to-width ratio. For example, in some embodiments, v-groove(s) may have an aspect ratio of less than or equal to 2, less than or equal to 1, or less than or equal to 0.5, and/or greater than or equal to 0.1, greater than or equal to 0.2, or greater than or equal to 0.3. Combinations of the above-referenced ranges are also possible (e.g., between or equal to 0.1 and 2, between or equal to 0.2 and 1). Other ranges are also possible.

In some embodiments, at least a portion of at least some channel(s) have a cross-section comprising a substantially triangular portion and a second portion opening into the substantially triangular portion and extending below the substantially triangular portion relative to the surface of the channel. In some embodiments, the second portion has a diameter (e.g., an average diameter) significantly smaller than an average diameter of the substantially triangular portion. Referring again to FIG. 5, in some embodiments, at least a portion of at least some of channels 102 have a cross-section comprising a substantially triangular portion 101 and a second portion 103 opening into substantially triangular portion 101 and extending below substantially triangular portion 101 relative to surface 105 of the channel, wherein second portion 103 has a diameter 107 significantly smaller than an average diameter 109 of substantially triangular portion 101. In some such cases, the second portion of a channel having a significantly smaller diameter than that of the average diameter of the substantially triangular portion of the channel can result in the substantially triangular portion being accessible to the roller of the apparatus and deformed portions of the surface layer, but the second portion being inaccessible to the roller and deformed portions of the surface layer. For example, referring again to FIG. 5, substantially triangular portion 101 of channel 102 is accessible to a roller (not pictured) and deformed portions of surface layer 106, while second portion 103 is inaccessible to the roller and deformed portions of surface layer 106, in accordance with certain embodiments. In some such cases, a seal with the surface layer 106 cannot be achieved in portions of the channel 102 having a second portion 103, because fluid can still move freely in second portion 103, even when surface layer 106 is deformed by a roller such that it fills substantially triangular portion 101 but not second portion 103. In some embodiments, a portion along a length of a channel may have both a substantially triangular portion and a second portion (“deep section”), while a different portion along the length of the channel has only the substantially triangular portion. In some such embodiments, when the apparatus (e.g., roller) engages with the portion having both a substantially triangular portion and a second portion (deep section), pump action is not started, because a seal with the surface layer is not achieved. However, as the apparatus engages along the length direction of the channel, when the apparatus deforms the surface layer at the portion of the channel having only a substantially triangular section, pump action begins because the lack of second portion (deep section) at that portion allows for a seal (and consequently a pressure differential) to be created. Therefore, in some cases, the presence and absence of deep sections along the length of the channels of the cartridge can allow for control of which portions of the channel are capable of undergoing pump action upon engagement with the apparatus.

The inclusion of such “deep sections” as second portions of at least some of the channels of the cartridge may contribute to any of a variety of potential benefits. For example, such deep sections (e.g., second portion 103) may, in some cases, contribute to a reduction in pump volume in peristaltic pumping processes. In some such cases, pump volume can be reduced by a factor of two or more for higher volume resolution. In some cases, such deep sections may also provide for a well-defined starting point for the pump volume that is not determined by where the roller lands on the channel. For example, the interface between a portion of a channel having both a substantially triangular portion and a second portion (deep section) and a portion of a channel having only a substantially triangular portion can, in some cases, be used as a well-defined starting point for the pump volume, because only fluid occupying the volume of the latter channel portion can be pumped. In some cases, where the rollers lands on the channel may have some error associated depending on any of a variety of factors, such as cartridge registration. The inclusion of deep sections may, in some cases, reduce or eliminate variations in pump volume associated with such error.

As used herein, an average diameter of a substantially triangular portion of a channel may be measured as an average over the z-axis from the vertex of the substantially triangular portion to the surface of the channel.

### SCODA

SCODA can involve providing a time-varying driving field component that applies forces to particles in some medium in combination with a time-varying mobility-altering field component that affects the mobility of the particles in the medium. The mobility-altering field component is correlated with the driving field component so as to provide a time-averaged net motion of the particles. SCODA may be applied to cause selected particles to move toward a focus area.

In one embodiment of SCODA based purification, described herein as electrophoretic SCODA, time varying electric fields both provide a periodic driving force and alter the drag (or equivalently the mobility) of molecules that have a mobility in the medium that depends on electric field strength, e.g. nucleic acid molecules. For example, DNA molecules have a mobility that depends on the magnitude of an applied electric field while migrating through a sieving matrix such as agarose or polyacrylamide. By applying an appropriate periodic electric field pattern to a separation matrix (e.g. an agarose or polyacrylamide gel) a convergent velocity field can be generated for all molecules in the gel whose mobility depends on electric field. The field-dependent mobility is a result of the interaction between a repeating DNA molecule and the sieving matrix, and is a general feature of charged molecules with high conformational entropy and high charge to mass ratios moving through sieving matrices. Since nucleic acids tend to be the only molecules present in most biological samples that have both a high conformational entropy and a high charge to mass ratio, electrophoretic SCODA based purification has been shown to be highly selective for nucleic acids.

The ability to detect specific biomolecules in a sample has wide application in the field of diagnosing and treating disease. Research continues to reveal a number of biomarkers that are associated with various disorders. Exemplary biomarkers include genetic mutations, the presence or absence of a specific protein, the elevated or reduced expression of a specific protein, elevated or reduced levels of a specific RNA, the presence of modified biomolecules, and the like. Biomarkers and methods for detecting biomarkers are potentially useful in the diagnosis, prognosis, and monitoring the treatment of various disorders, including cancer, disease, infection, organ failure and the like.

The differential modification of biomolecules in vivo is an important feature of many biological processes, including development and disease progression. One example of differential modification is DNA methylation. DNA methylation involves the addition of a methyl group to a nucleic acid. For example a methyl group may be added at the 5′ position on the pyrimidine ring in cytosine. Methylation of cytosine in CpG islands is commonly used in eukaryotes for long term regulation of gene expression. Aberrant methylation patterns have been implicated in many human diseases including cancer. DNA can also be methylated at the 6 nitrogen of the adenine purine ring.

Chemical modification of molecules, for example by methylation, acetylation or other chemical alteration, may alter the binding affinity of a target molecule and an agent that binds the target molecule. For example, methylation of cytosine residues increases the binding energy of hybridization relative to unmethylated duplexes. The effect is small. Previous studies report an increase in duplex melting temperature of around 0.7° C. per methylation site in a 16 nucleotide sequence when comparing duplexes with both strands unmethylated to duplexes with both strands methylated.

### Affinity SCODA

SCODAphoresis is a method for injecting biomolecules into a gel, and preferentially concentrating nucleic acids or other biomolecules of interest in the center of the gel. SCODA may be applied, for example, to DNA, RNA and other molecules. Following concentration, the purified molecules may be removed for further analysis. In one specific embodiment of SCODAphoresis-affinity SCODA-binding sites which are specific to the biomolecules of interest may be immobilized in the gel. In doing so one may be able generate a non-linear motive response to an electric field for biomolecules that bind to the specific binding sites. One specific application of affinity SCODA is sequence-specific SCODA. Here, oligonucleotides may be immobilized in the gel allowing for the concentration of only DNA molecules which are complementary to the bound oligonucleotides. All other DNA molecules which are not complementary may focus weakly or not at all and can therefore be washed off the gel by the application of a small DC bias.

SCODA-based transport is a general technique for moving particles through a medium by first applying a time-varying forcing (i.e., driving) field to induce periodic motion of the particles and superimposing on this forcing field a time-varying perturbing field that periodically alters the drag (or equivalently the mobility) of the particles (i.e., a mobility-altering field). Application of the mobility-altering field is coordinated with application of the forcing field such that the particles will move further during one part of the forcing cycle than in other parts of the forcing cycle.

By varying the drag (i.e., mobility) of the particle at the same frequency as the external applied force, a net drift can be induced with zero time-averaged forcing. An appropriate choice of driving force and drag coefficients that vary in time and space can generate a convergent velocity field in one or two dimensions. A time varying drag coefficient and driving force can be utilized in a real system to specifically concentrate (i.e., preferentially focus) only certain molecules, even where the differences between the target molecule and one or more non-target molecules are very small, e.g. molecules that are differentially modified at one or more locations, or nucleic acids differing in sequence at one or more bases.

An affinity matrix can be generated by immobilizing an agent with a binding affinity to the target molecule (i.e., a probe) in a medium. Using such a matrix, operating conditions can be selected where the target molecules transiently bind to the affinity matrix with the effect of reducing the overall mobility of the target molecule as it migrates through the affinity matrix. The strength of these transient interactions is varied over time, which has the effect of altering the mobility of the target molecule of interest. SCODA drift can therefore be generated. This technique is called affinity SCODA, and is generally applicable to any target molecule that has an affinity to a matrix.

Affinity SCODA can selectively enrich for nucleic acids based on sequence content, with single nucleotide resolution. In addition, affinity SCODA can lead to different values of k for molecules with identical DNA sequences but subtly different chemical modifications such as methylation. Affinity SCODA can therefore be used to enrich for (i.e., preferentially focus) molecules that differ subtly in binding energy to a given probe, and specifically can be used to enrich for methylated, unmethylated, hypermethylated, or hypomethylated sequences.

Exemplary media that can be used to carry out affinity SCODA include any medium through which the molecules of interest can move, and in which an affinity agent can be immobilized to provide an affinity matrix. In some embodiments, polymeric gels including polyacrylamide gels, agarose gels, and the like are used. In some embodiments, microfabricated/microfluidic matrices are used.

Exemplary operating conditions that can be varied to provide a mobility altering field include temperature, pH, salinity, concentration of denaturants, concentration of catalysts, application of an electric field to physically pull duplexes apart, or the like.

Exemplary affinity agents that can be immobilized on the matrix to provide an affinity matrix include nucleic acids having a sequence complementary to a nucleic acid sequence of interest, antibodies specific for modified or unmodified molecules, nucleic acid aptamers specific for modified or unmodified molecules, other molecules or chemical agents that preferentially bind to modified or unmodified molecules, or the like.

The affinity agent may be immobilized within the medium in any suitable manner. For example, where the affinity agent is an oligonucleotide, the oligonucleotide may be covalently bound to the medium, acrydite modified oligonucleotides may be incorporated directly into a polyacrylamide gel, the oligonucleotide may be covalently bound to a bead or other construct that is physically entrained within the medium, or the like.

Where the affinity agent is a small molecule that interacts with the molecule of interest, the affinity agent may be covalently coupled to the medium in any suitable manner.

One embodiment of affinity SCODA is sequence-specific SCODA. In sequence specific SCODA, the target molecule is or comprises a nucleic acid molecule having a specific sequence, and the affinity matrix contains immobilized oligonucleotide probes that are complementary to the target nucleic acid molecule. In some embodiments, sequence specific SCODA is used both to separate a specific nucleic acid sequence from a sample, and to separate and/or detect whether that specific nucleic acid sequence is differentially modified within the sample. In some such embodiments, affinity SCODA is conducted under conditions such that both the nucleic acid sequence and the differentially modified nucleic acid sequence are concentrated by the application of SCODA fields. Contaminating molecules, including nucleic acids having undesired sequences, can be washed out of the affinity matrix during SCODA focusing. A washing bias can then be applied in conjunction with SCODA focusing fields to separate the differentially modified nucleic acid molecules as described below by preferentially focusing the molecule with a higher binding energy to the immobilized oligonucleotide probe.

Sequences

SEQ ID NO: 1 (Adaptor Design A)
CTGTCTCTTATACACATCTGACGCTGCCGACGATTTTCCTCCTCCTCCIT
TITTITTITTGTCTCGTGGGCTCGGAGATGTGTATAAGAGACAG
SEQ ID NO: 2 (Index primer A)
GACGCTGCCGACGA
SEQ ID NO: 3 (Index primer B)
GTCTCGTGGGCTCGG
SEQ ID NO: 4 (P-B Primer)
TTTTCCTCCTCCTCCITTITTITTITT
SEQ ID NO: 5 (Tn5 mosaic end)
AGATGTGTATAAGAGACAG
SEQ ID NO: 6 (Tn5 mosaic end - reverse)
CTGTCTCTTATACACATCT
SEQ ID NO: 7 (QsiTN5a ver1)
SEQ ID NO: 8 (Adaptor Design, wherein ‘X’ can be
thymine, inosine, uridine, 5-methylcytosine,
isoguanine, 2-thiouracil, and 4-thiouracil)
CTGTCTCTTATACACATCTGACGCTGCCGACGATTTTCCTCCTCCTCCXX
XXXXXXXXXXGTCTCGTGGGCTCGGAGATGTGTATAAGAGACAG

### Definitions

In the specification, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as “comprises” and “comprising,” are to be construed in an open, inclusive sense (i.e., as “including, but not limited to”).

Unless defined otherwise, all technical and scientific terms used herein have the same meaning as is commonly understood by one of skill in the art to which this invention belongs. As used in the specification and claims, the singular form “a”, “an”, and “the” include plural references unless the context clearly dictates otherwise.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

### Further Aspects of the Invention

Aspects of the exemplary embodiments and examples described above may be combined in various combinations and subcombinations to yield further embodiments of the invention. To the extent that aspects of the exemplary embodiments and examples described above are not mutually exclusive, it is intended that all such combinations and subcombinations are within the scope of the present invention. It will be apparent to those of skill in the art that embodiments of the present invention include a number of aspects. Accordingly, the scope of the claims should not be limited by the preferred embodiments set forth in the description and examples, but should be given the broadest interpretation consistent with the description as a whole.

What is claimed is:

1. A nucleic acid adaptor, comprising:
a double-stranded transposase recognition sequence;
a first primer-binding sequence; and
a pair of index primer-binding sequences comprising a first index primer-binding sequence and a second index primer-binding sequence.

2. The nucleic acid adaptor of claim 1, wherein the double-stranded transposase recognition sequence is a double-stranded Tn5 transposase recognition sequence.

3. The nucleic acid adaptor of claim 1, wherein the double-stranded transposase recognition sequence is a mosaic end.

4. The nucleic acid adaptor of claim 1, wherein the double-stranded transposase recognition sequence is 15-25 nucleotides in length, optionally 19 nucleotides in length.

5. The nucleic acid adaptor of claim 1, wherein:
(a) a first strand of the double-stranded transposase recognition sequence comprises the nucleic acid sequence set forth in SEQ ID NO: 5; or
(b) a first strand of the double-stranded transposase recognition sequence comprises the nucleic acid sequence set forth in SEQ ID NO: 6; or
(c) a first strand of the double-stranded transposase recognition sequence comprises the nucleic acid sequence set forth in SEQ ID NO: 5, and a second strand of the double-stranded transposase recognition sequence comprises the nucleic acid sequence set forth in SEQ ID NO: 6.

6. The nucleic acid adaptor of claim 1, wherein the first primer-binding sequence comprises one or more non-standard nucleotides selected from the group consisting of: inosine, uridine, 5-methylcytosine, isoguanine, 2-thiouracil, and 4-thiouracil.

7. The nucleic acid adaptor of claim 1, wherein the first primer-binding sequence comprises four inosine nucleotides.

8. The nucleic acid adaptor of claim 1, wherein the first primer-binding sequence comprises the nucleic acid sequence set forth in SEQ ID NO: 4.

9. The nucleic acid adaptor of claim 1, wherein the first index primer-binding sequence comprises the nucleic acid sequence set forth in SEQ ID NO: 2 and/or SEQ ID NO: 3.

10. The nucleic acid adaptor of claim 1, wherein the first primer-binding sequence is configured for use in a first sequencing instrument, and the pair of index primer-binding sequences are configured for use in a second sequencing instrument.

11. The nucleic acid adaptor of claim 10, wherein the first sequencing instrument is a long-read sequencing instrument.

12. The nucleic acid adaptor of claim 10, wherein the first sequencing instrument is a high-throughput sequencing instrument.

13. The nucleic acid adaptor of claim 1, comprising the nucleic acid sequence set forth in SEQ ID NO: 1.

14. A circular nucleic acid comprising one or two nucleic acid adaptors, wherein at least one of the one or two nucleic acid adaptors is the nucleic acid adaptor of claim 1.

15. The circular nucleic acid of claim 14, comprising two nucleic acid adaptors on opposite sides of the circular nucleic acid.

16. The circular nucleic acid claim 14, comprising two identical nucleic acid adaptors.

17. A method of preparing a nucleic acid library for sequencing, the method comprising:
(i) contacting a target nucleic acid with a transposon and the nucleic acid adaptor of claim 1 to generate a transposase-mediated fragment;
(ii) contacting the transposase-mediated fragment with one or more enzymes necessary to fill the gaps and circularize the fragment.

18. The method of claim 17, wherein the transposon is a member of the Tn5 transposase family of proteins.

19. The method of claim 17, wherein the one or more enzymes necessary to fill the gaps and circularize the fragment comprise a polymerase and a ligase.

20. The method of claim 17, wherein the target nucleic acid is from a biological sample.
